(19) United States
(12) Patent Application Publication
(10) Pub. No.: US 2022/0079779 A1
(43) Pub. Date: Mar. 17, 2022

(54) VOLUME-ADAPTABLE BREAST PROSTHESIS

(71) Applicant: AMOENA MEDIZIN-ORTHOPÄDIE-TECHNIK GMBH, Raubling (DE)

(72) Inventors: Nils STELTER, Frasdorf (DE); Norbert SCHÖNERT, Raubling (DE)

(21) Appl. No.: 17/421,439

(22) PCT Filed: Jan. 9, 2020
(86) PCT No.: PCT/EP2020/050391

§ 371 Date: Jul. 8, 2021

(30) Foreign Application Priority Data
Jan. 10, 2019 (DE) 10 2019 100 484.5

(51) Int. Cl. A61F 2/52

(52) U.S. Cl. CPC A61F 2/52; A61F 2002/523

(57) ABSTRACT

The invention relates to a breast prosthesis having an adaptable volume, wherein the breast prosthesis has a first shell body, a second shell body peripherally connected thereto, and a fluid space that is arranged between the inner surfaces of the shell bodies and that is filled with a fluid for volume adaptation, and wherein a medium that reduces the adhesive tendency of the oppositely disposed inner surfaces of the shell bodies is located in the fluid space in addition to the fluid for the volume adaptation.

The invention relates to a breast prosthesis or a breast epithesis having an adaptable volume.

Breast prostheses are worn after surgical breast removals. Demands on breast prostheses in particular include a shape and feel that come as close as possible to the natural breast as well as a high comfort in wear.

To be able to accomplish being able to adapt the volume of such a prosthesis to the individual needs of the wearer that result from the size of the still healthy breast, if present, or from personal well-being without expensive custom-made solutions, it has already been proposed in the prior art to provide breast prostheses whose volume can be retroactively adapted. EP 2 554 138 A1 or EP 0 824 001 A2 can be named as examples for this.

It is the object of the invention to provide a volume-adaptable breast prosthesis having improved properties.

Against this background, the invention relates to a breast prosthesis having an adaptable volume, wherein the breast prosthesis has a first shell body, a second shell body peripherally connected thereto, and a fluid space that is arranged between the inner surfaces of the shell bodies and that is filled with a fluid for volume adaptation, and wherein a medium that reduces the adhesive tendency of the oppositely disposed inner surfaces of the shell bodies is located in the fluid space in addition to the fluid for the volume adaptation.

The subsequent volume adaptation can be impeded in that the inner surfaces of the shell bodies adhere to one another. A permanent deformation of the breast prosthesis by such an adhesion would also have to be feared. The invention therefore provides reducing the tendency for such an unwanted adhesion.

The shell bodies are typically themselves flexible in order, on the one hand, to be able to satisfy the demands on haptics and comfort in wear and, on the other hand, to permit an expansion of the fluid space volume. The peripheral connection between the shell bodies typically takes place by adhesive bonding or welding along a common peripheral surface.

The first shell body, the second shell, body, or both shell bodies are film bags filled with a deformable material, with the film from which the film bags are produced preferably being a plastic film. The deformable material is preferably a crosslinked two-component silicone rubber. The film bags can be produced from two film pieces that are connected, preferably welded, to one another, along the common peripheral surface.

Provision is made in an embodiment that the breast prosthesis further comprises a valve tube that is composed of a flexible material, and preferably a plastic material, and that reaches into the fluid space from the outside in the connection region between the shell bodies and projects beyond the connection region into the fluid space. The valve tube generally has a check valve such as a flutter valve and serves to enable a subsequent filling and emptying of the fluid space, i.e. after the production. The tube can pass through the weld seam or adhesive seam in the radial direction between the film bags and can be adhered or welded between the shell bodies in this process.

The section of the valve tube projecting into the fluid space can be connected to neither or only to one of the shell bodies. The section of the valve tube projecting into the fluid space is therefore freely movable in the fluid space or is only connected to one shell body. The medium for reducing the adhesive tendency between the inner surfaces of the shell bodies preferably simultaneously produces a reduction of the adhesive tendency between the section of the valve tube projecting into the fluid space and the inner surfaces of the shell bodies to which it is not connected.

The fluid for the volume adaptation can be a gas, in particular air. The fluid for the volume adaptation can furthermore be a liquid, in particular a subsequently crosslinkable viscous liquid and further preferably a crosslinkable silicone fluid. Combinations are also conceivable.

The medium for reducing the adhesion tendency of the oppositely disposed inner surfaces of the shell bodies can be a liquid, in particular an oil and further preferably a silicone oil. In the event that both the fluid for the volume adaptation and the medium for reducing the adhesion tendency of the oppositely disposed inner surfaces of the shell bodies are respectively liquids, these liquids differ from one another.

The medium for reducing the adhesive tendency of the oppositely disposed inner surfaces of the shell bodies can furthermore be a powdery solid. The average particle size of the powder grains is preferably in the range between 1 nm to 1 mm.

The medium for reducing the adhesion tendency of the oppositely disposed inner surfaces of the shell bodies can optionally also be a gas. In the event that both the fluid for the volume adaptation and the medium for reducing the adhesion tendency of the oppositely disposed inner surfaces of the shell bodies are respectively gases, these gases differ from one another.

Combinations of said media for reducing the adhesion tendency of the oppositely disposed inner surfaces of the shell bodies are also used in further embodiments. A mixture of liquids and dispersed powdery solids can be used, for example, with the liquids and dispersed powdery solids being able to be formed as defined above.

Further details and advantages of the invention result from the following embodiment described with reference to the Figures. The Figures show:

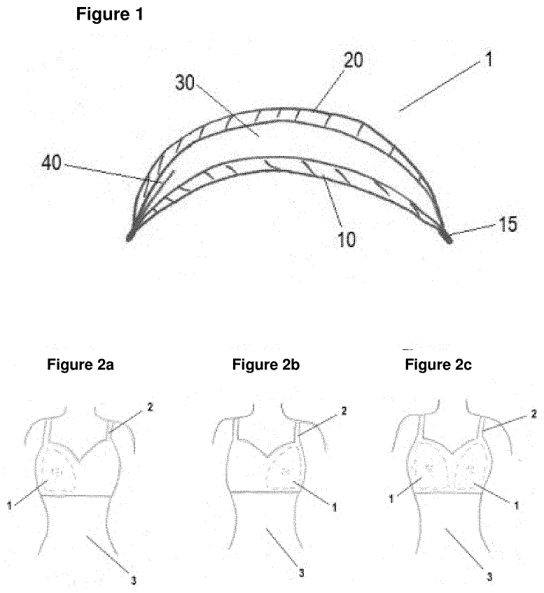

FIG. 1: a schematic representation of a volume-adaptable breast prosthesis in accordance with the invention;

FIG. 2: a schematic representation of a patient with a bra and a breast prosthesis on the right side, on the left side, and in both sides of the bra;

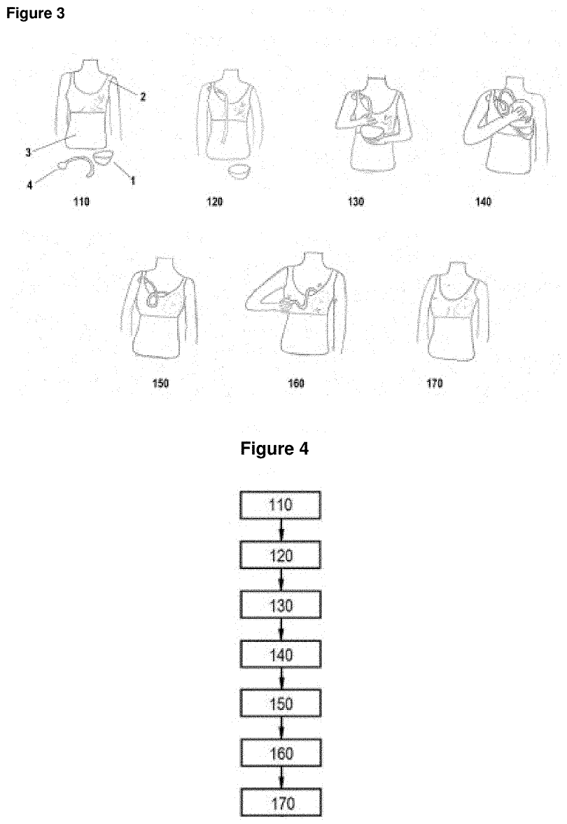

FIG. 3. an illustrative representation of a routine of a method for a volume adaptation of a breast prosthesis in accordance with the invention at the user side;

FIG. 4: an associated flowchart;

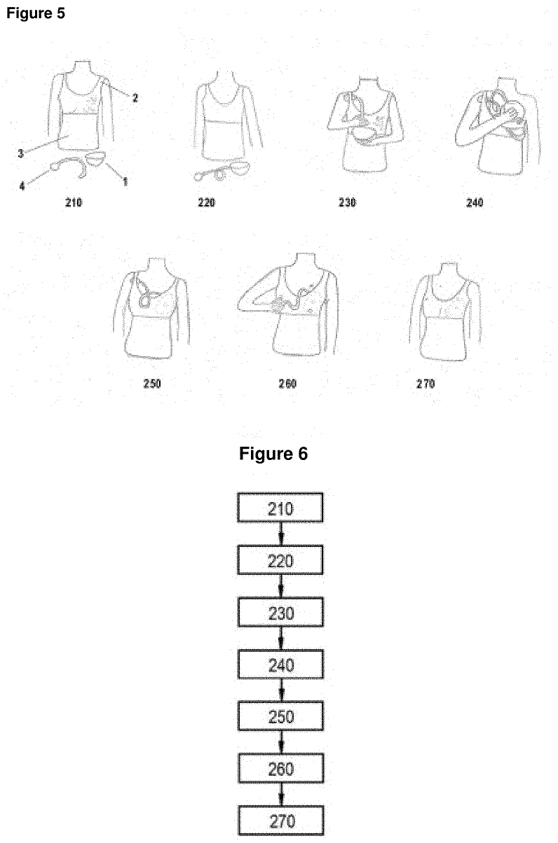

FIG. 5: an illustrative representation of a procedure of an alternative method for the volume adaptation of a breast prosthesis in accordance with the invention at the user side; and

FIG. 6: an associated flowchart.

The breast prosthesis 1 in accordance with the invention shown in FIG. 1 having an adaptable volume comprises a first shell body 10 at the lower side of the prosthesis 1 facing the wearer and a second shell body 20 peripherally connected thereto at the upper side of the prosthesis 1 facing away from the wearer. Both shell bodies 10 and 20 are film bags that are filled with a crosslinked two-component silicone rubber compound. The film bags are each produced from two plastic film pieces that are welded to one another along the common peripheral surface.

The shell bodies 10 and 20 are in turn connected along a peripheral weld seam 15 such that a fluid space 30 is formed between them that can, for example, be filled with air, but also with a liquid. The volume of the breast prosthesis 1 can be adapted by filling and emptying the fluid space 30.

To make a subsequent filling and emptying of the fluid space 30 possible, i.e. one taking place after the production, the breast prosthesis 1 comprises a valve tube 40 that is composed of a flexible plastic material and that comprises a flat flutter valve, that penetrates the weld seam 15 in the radial direction, and that is welded between the shell bodies 10 and 20 in this process. The valve tube 40 does not only reach up to the end of the weld seam 15, but projects freely, i.e. without being connected to one of the shell bodies 10 or 20, beyond the weld seam 15, into the fluid space 30. The section of the valve tube 40 projecting into the fluid space 40 is therefore freely movable in the fluid space 30.

To prevent a sticking together of the plastic inner surfaces of the two shell bodies 10 and 20 due, for example, to electrostatic interaction and thus to prevent an unwanted adhesion of the fluid space 30, the fluid space 30 is already filled with a smaller amount of silicone oil at the side of the manufacturer. A variable amount of air can additionally be blown into the fluid space 30 at the user side as the medium for the volume adaptation.

FIG. 2 shows a patient who is wearing a breast prosthesis 1 in accordance with the invention on the right side (FIG. 2a), on the left side (FIG. 2b), or on both sides (FIG. 2c). Provision is made here that the volume-adaptable breast prosthesis 1 can be placed between the skin and the cup of the bra 2. If the bra 2 has integrated pockets, the prosthesis 1 can also be placed therein. The volume-adaptable prosthesis 1 can be continuously increased or decreased in volume in this position by the user 3 or by a further person until the desired volume is reached, for instance based on the feeling of the user 3.

FIGS. 3-4 show an illustrative representation of a procedure of an embodiment variant of a method for volume adaptation and an associated flowchart. In a first step 110, the user 3 who is wearing a bra 2, prepares the volume-adaptable breast prosthesis 1 and a suitable pump accessory 4, for example comprising a pump, a tube, and a needle. In a second step 120, the pump accessory 4 is fixed at a suitable position between the bra 2 and the skin of the patient 3, for example is clamped between the straps of the bra 2 and the skin of the patient 3. In a further step 130, the free end of the pump accessory 4, that is, for example, the needle, is connected to the volume-adaptable breast prosthesis 1 in that, for example, the needle is placed into the valve tube. In a next step 140, the bra 2 is opened and the breast area exposed at the side of the bra 2 at which the breast prosthesis 1 should be inserted (in this case at the right from the viewpoint of the wearer). The volume-adaptable breast prosthesis 1 is then placed between the skin of the patient 3 and the cup of the bra 2 in step 150. In the case of a bra 2 having integrated pockets, the breast prosthesis 1 can also be placed therein. The bra is then closed again and brought into the normal position of wear. Subsequently, in a step 160, the clamped region of the pump accessory 4 is removed using a free hand of the user 3 so that the user 3 can increase or decrease the volume of the breast prosthesis by means of the pump accessory 4. The method ends at step 170 with the finalizing of the increasing or decreasing procedure when the desired size has been reached.

FIGS. 5-6 show an illustrative representation of a routine of a further embodiment variant of a method for volume adaptation and an associated flowchart. In a first step 210 of the embodiment variant, the user 3 who is wearing a suitable bra 2, prepares the volume-adaptable breast prosthesis 1 and a suitable accessory 4 comprising a pump, a tube, and a cannula. In second step 220, the user 3 connects the pump accessory 4 to the breast prosthesis 1, for example by inserting the needle into the valve tube. The following steps 230-270 correspond to steps 130-170 of the embodiment in accordance with FIGS. 3-4.

The volume adaptation by means of the suitable pump accessory 4 take place as part of both method procedures shown, for example also by the user 3 herself in front of a mirror without any aiding person.

1. A breast prosthesis having an adaptable volume, wherein the breast prosthesis has a first shell body, a second shell body peripherally connected thereto, and a fluid space arranged between the inner surfaces of said shell bodies that is filled with a fluid for volume adaptation, wherein a medium that reduces the adhesive tendency of the oppositely disposed inner surfaces of the shell bodies is located in the fluid space in addition to the fluid for the volume adaptation.

2. A breast prosthesis in accordance with claim 1, wherein the first shell body, the second shell, body, or both shell bodies is/are film bags filled with a deformable material, with the film from which the film bags are produced preferably being a plastic film.

3. A breast prosthesis in accordance with claim 1, wherein the breast prosthesis further comprises a valve tube composed of a flexible material, and preferably a plastic material, and reaches into the fluid space from the outside in the connection region between the shell bodies and projects beyond the connection region into the fluid space.

4. A breast prosthesis in accordance with claim 3, wherein the section of the valve tube projecting into the fluid space is connected to neither of the shell bodies or only to one of the shell bodies.

5. A breast prosthesis in accordance with claim 1, wherein the fluid for volume adaptation is a gas, in particular air.

6. A breast prosthesis in accordance with claim 1, wherein the fluid for the volume adaptation is a liquid, in particular a subsequently crosslinkable viscous liquid and further preferably a crosslinkable silicone fluid.

7. A breast prosthesis in accordance with claim 1, wherein the medium for reducing the adhesion tendency of the oppositely disposed inner surfaces of the shell bodies can be a liquid, in particular an oil and preferably a silicone oil.

8. A breast prosthesis in accordance with claim 1, wherein the medium for reducing the adhesive tendency of the oppositely disposed inner surfaces of the shell bodies is a powdery solid.

9. A breast prosthesis in accordance with claim 2, wherein the breast prosthesis further comprises a valve tube composed of a flexible material, and preferably a plastic material, and reaches into the fluid space from the outside in the connection region between the shell bodies and projects beyond the connection region into the fluid space.

10. A breast prosthesis in accordance with claim 9, wherein the section of the valve tube projecting into the fluid space is connected to neither of the shell bodies or only to one of the shell bodies.

11. A breast prosthesis in accordance with claim 10, wherein the fluid for volume adaptation is a gas, in particular air.

12. A breast prosthesis in accordance with claim 9, wherein the fluid for volume adaptation is a gas, in particular air.

13. A breast prosthesis in accordance with claim 4, wherein the fluid for volume adaptation is a gas, in particular air.

14. A breast prosthesis in accordance with claim 3, wherein the fluid for volume adaptation is a gas, in particular air.

15. A breast prosthesis in accordance with claim 2, wherein the fluid for volume adaptation is a gas, in particular air.

16. A breast prosthesis in accordance with claim 15, wherein the fluid for the volume adaptation is a liquid, in particular a subsequently crosslinkable viscous liquid and further preferably a crosslinkable silicone fluid.

17. A breast prosthesis in accordance with claim 14, wherein the fluid for the volume adaptation is a liquid, in particular a subsequently crosslinkable viscous liquid and further preferably a crosslinkable silicone fluid.

18. A breast prosthesis in accordance with claim 13, wherein the fluid for the volume adaptation is a liquid, in particular a subsequently crosslinkable viscous liquid and further preferably a crosslinkable silicone fluid.

19. A breast prosthesis in accordance with claim 12, wherein the fluid for the volume adaptation is a liquid, in particular a subsequently crosslinkable viscous liquid and further preferably a crosslinkable silicone fluid.

20. A breast prosthesis in accordance with claim 11, wherein the fluid for the volume adaptation is a liquid, in particular a subsequently crosslinkable viscous liquid and further preferably a crosslinkable silicone fluid.
